(19) United States
(12) Patent Application Publication
(10) Pub. No.: US 2017/0007171 A1
(43) Pub. Date: Jan. 12, 2017

(54) Apparatus, System, and Method for Measuring Lymphatic Pressure and Performance in Vivo

(71) Applicant: GEORGIA TECH RESEARCH CORPORATION, Atlanta, GA (US)

(72) Inventors: Michael J. Weiler, Atlanta, GA (US); James Brandon Dixon, Atlanta, GA (US); Tyler S. Nelson, Atlanta, GA (US); Ryan E. Akim, Atlanta, GA (US)

(21) Appl. No.: 15/115,104

(22) PCT Filed: Feb. 9, 2015
(86) PCT No.: PCT/US15/15055

§ 371 Date: Jul. 28, 2016

Related U.S. Application Data
(60) Provisional application No. 61/936,926, filed on Feb. 7, 2014.

(51) Int. Cl. A61B 5/00; A61M 5/00; A61B 5/022; A61B 5/03

(52) U.S. Cl. CPC A61B 5/418; A61B 5/03; A61B 5/004; A61B 5/0071; A61B 5/0082; A61M 5/007; A61B 5/02233

(57) ABSTRACT

Methods for measuring lymphatic performance are provided herein. Lymphatic pressure is measured by introducing a near infrared flourophore into the limb of a mammal, detecting the lymph flow with near infrared lights and sensors, occluding lymph flow with a pressure cuff, and then noting the pressure at which lymph flow returns. Lymphatic performance is derived by comparing lymphatic pressure measurements taken at multiple points along a mammal's limb, and by measuring the rate at which lymph is emptied upstream from occluded vessels.

### CROSS REFERENCE TO RELATED APPLICATIONS

This application claims the benefit of U.S. Provisional Patent Application No. 61/936,926, filed Feb. 7, 2014, the contents of which are hereby fully incorporated herein by reference.

### FIELD OF THE INVENTION

The present invention relates generally to an apparatus, system and method for measuring lymphatic performance. In exemplary embodiments, the present invention provides a method for measuring lymphatic performance by determining lymphatic pressure per unit length (PPL), by determining emptying rate, or a combination thereof. In certain embodiments, the method isolates intrinsic contractility from extrinsic factors, permitting an improved understanding of lymphatic performance.

### BACKGROUND OF THE INVENTION

The mammalian lymphatic system operates to return interstitial fluids from the extremities back into the circulatory system by way of a network of contractile vessels (“lymph vessels”) and nodes (“lymph nodes”). A disruption in lymphatic system performance can lead to a buildup of fluid in the limbs, known as lymphedema, which can result in permanent and debilitating swelling. Lymphatic flow is driven by intrinsic contractility (contractions of the vasculature itself) and extrinsic factors (skeletal muscle contraction, interstitial fluid pressure, gravity, nodal resistance, fluid backpressure, etc.). It is widely thought that the intrinsic contractility of the vessels may be the most important factor in the onset and progression of lymphedema.

Over the past two decades, research into lymphatic performance has focused chiefly on the velocities and flow rates of lymph within lymph vessels, resulting in advances in the imaging techniques available to quantify these metrics. Near infrared imaging has grown in favor because it provides the necessary spatial and temporal resolution to visualize lymph flow, and also presents low radiological exposure to the test subject.

Near infrared imaging methods generally require the use of four separate components: light source, camera, near infrared flourophore (“contrast agent”), and a computer. See, for example, U.S. Patent Application Publication No. 2013/0267843 to Sevick-Muraca et al. The light source is typically a low powered laser, which outputs in the near infrared spectrum, and is equipped with lenses and other optics to provide a sufficient but not harmful amount of light.

The camera chosen must be able to capture images at a known rate, and is typically equipped with lenses and filters so it only senses light in the near infrared spectrum. The contrast agents typically used are excited by near infrared light of one wavelength and emit light at a different wavelength, which is also in the near infrared spectrum. The computer controls the light and the camera, and conducts processing to determine the presence or absence of contrast agent, as signaled by a change in light intensity of the images. The light and camera are trained on a limb of the mammal for which lymphatic measurement is to be conducted. At a point on the limb that is distal from the light source and camera, the contrast agent is injected into the limb, from which the lymphatic vessels will naturally draw the contrast agent and begin to transport it towards the mammal's trunk. As the contrast agent progresses through the lymph vessels, it will eventually pass into and through the region on which the light source and camera are focused. At this point, the contrast agent will be excited by the light source and emit near infrared light at a different wavelength from the light source, and this new wavelength will be detected by the camera. Images are captured at regular intervals, which taken together show the progress of the contrast agent through the lymph vessels, and these can be used to determine the velocity and flow rate of the lymph within the lymph vessels.

Modifications of this large scale setup have been disclosed, including the use of a pressure cuff to obstruct lymph flow, as disclosed in U.S. Patent Application Publication No. 2012/0123254 to Unno (“Unno”). In that arrangement, the near infrared laser and camera are directed through a transparent pressure cuff onto the subject's limb. A contrast agent is injected at a location remote from the pressure cuff, and the lymphatic vessels naturally draw the contrast agent toward the location off the pressure cuff. When the infrared camera senses the presence of the contrast agent through the transparent pressure cuff, the pressure cuff is inflated to obstruct the lymph flow, and then slowly deflated while the near infrared laser and camera is used to detect the return of flow, which occurs when the lymphatic pressure is greater than the pressure exerted by the cuff Unno additionally discloses an integrated pressure cuff unit, which uses LEDs instead of lasers for providing near infrared light, and uses photo sensors instead of infrared cameras to detect the presence of contrast agent. Notably, both systems disclosed in Unno measure lymphatic pressure at a single point along a mammal's limb.

While ability to measure and quantify lymphatic pressure and velocity has therefore been demonstrated, known techniques measure a combination of intrinsic contractility and extrinsic factors. It is not possible using known techniques to isolate the intrinsic contractility of the lymphatic vessels from other extrinsic factors, which limits the understanding of lymphatic performance.

Accordingly, there remains a need to perform lymphatic performance in a manner that isolates intrinsic contractility.

### SUMMARY OF THE INVENTION

The present invention is directed to methods for measuring lymphatic performance, particularly in a minimally invasive in vivo operation performed on the limb of a mammal. In exemplary embodiments, the method involves measuring lymphatic performance by determining lymphatic pressure at a plurality of locations. Lymphatic pressure can be measured using an apparatus comprising an integrated inflatable cuff, light source, light sensor, inflation means, deflation means, and pressure transducer, or can be measured using the device described in Unno.

In a first aspect, the present invention is a method for measuring lymphatic performance in a mammal by calculating lymphatic pressure per unit length (PPL), the method comprising (a) placing an inflatable cuff around the circumference of a limb of the mammal at a cuff location, (b) illuminating the limb with at least one light source at an interrogation region, wherein the interrogation region is proximal to or co-located with the cuff location, wherein the at least one light source is operable to emit light at an emission wavelength, (c) imaging the interrogation region with at least one light sensor, wherein the at least one light sensor is operable to sense light at a detection wavelength, (d) introducing a volume of a contrast agent into the limb at an injection location, wherein the injection location is distal to the cuff location, wherein the contrast agent is drawn to the cuff location and excited by light at the emission wavelength, producing florescence, (e) detecting the florescence in the interrogation region with the at least one light sensor, (f) inflating said inflatable cuff to a pressure sufficient to block lymphatic flow, (g) detecting the decrease in florescence in the interrogation region with the at least one light sensor, (h) gradually deflating the inflatable cuff, (i) detecting the return of florescence in the interrogation region with the at least one light sensor, (j) determining the pressure at which florescence returned, providing a first lymphatic pressure; (k) measuring lymphatic pressure at a second location along the mammal's limb, proximal to the first location, according to the above steps except that no further contrast agent is required, to provide a second lymphatic pressure; and (1) determining the difference between the first and second lymphatic pressures and dividing by the distance between the first and second location to provide a change in lymphatic pressure per unit length (PPL).

The inflatable cuff may be any inflatable cuff suitable for use in blocking lymphatic flow. In an exemplary embodiment, the inflatable cuff used to determine the first lymphatic pressure is the same as the inflatable cuff used to determine the second lymphatic pressure. In exemplary embodiments, the inflatable cuff is relocated from the first location to the second location. In exemplary embodiments, the inflatable cuff used to determine the first lymphatic pressure is different than the inflatable cuff used to determine the second lymphatic pressure. In an exemplary embodiment, the inflatable cuff is integrated with one or more components selected from the group consisting of light source, light sensor, inflation means, deflation means, and pressure transducer.

In a second aspect, the present invention provides a method for measuring lymphatic performance by determining an emptying rate, comprising (a) placing a lymphatic occlusion means around the circumference of a limb of a mammal at a cuff location, (b) illuminating said limb with at least one light source at an interrogation region, wherein said interrogation region is proximal to or co-located with said cuff location, wherein said at least one light source is operable to emit light at an emission wavelength, (c) imaging the interrogation region with at least one light sensor, wherein said at least one light sensor is operable to detect light at a detection wavelength, (d) introducing a volume of a contrast agent into said limb at an injection location, wherein said injection location is distal to said cuff location, wherein said contrast agent is wherein said contrast agent is drawn to the cuff location and excited by light at the emission wavelength, (e) detecting the presence of florescence in the interrogation region with the at least one light sensor, (f) occluding the lymphatics with a lymphatic occlusion means, (h) determining florescence in the interrogation region with the at least one light sensor at a first time to provide a first level of florescence, (g) determining florescence in the interrogation region with the at least one light sensor at a second time to provide a second level to provide a second level of florescence, and (h) dividing the difference between the first level of florescence and the second level of florescence by the difference between the first time and the second time to provide an emptying rate.

The lymphatic occlusion means may be any device which has the effect of blocking lymphatic flow through the interrogation region. The inflatable cuff described previously would accomplish this, as would a simple tourniquet.

### BRIEF DESCRIPTION OF THE FIGURES

FIG. 1 illustrates a first example of the apparatus disclosed in of Unno. A transparent pressure cuff 11 is placed on a limb, and a camera unit 21 is used to detect the flow of contrast agent which is illuminated by a near infrared light source (not pictured). In this embodiment, the camera and light source are components that are separate from, rather than integrated with, the pressure cuff

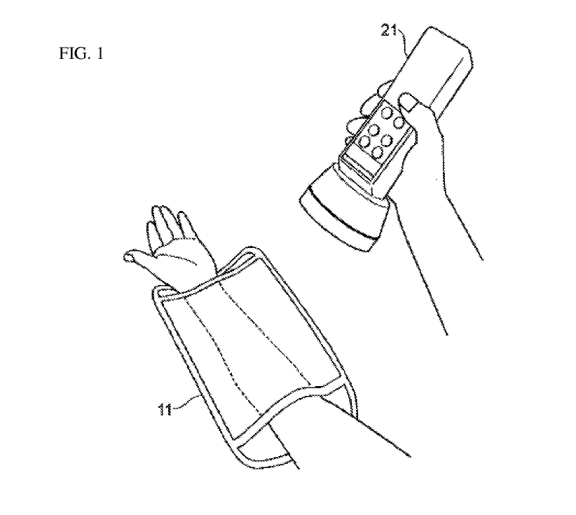

FIG. 2 reproduces FIG. 11 from Unno, which shows one embodiment of a lymphatic pressure measuring apparatus. A pressure cuff 40 includes a first detection unit 42 and a second detection unit 43. The first detection unit and second detection unit include a line of LEDs (42a and 43a) and a line sensor (42b and 43b). The first detection unit 42 is used to trigger the pressure cuff 40 to contract, and the second detection unit 43 determines when flow has resumed. In this embodiment, the camera and light source are components that are integrated with the pressure cuff.

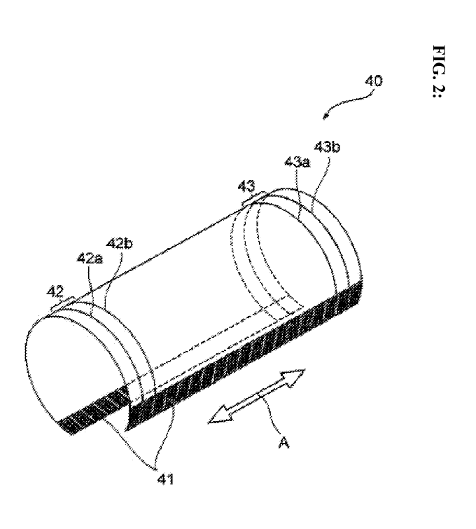

FIG. 3 shows the result of current research conducted by the inventors. Pressure measurements were taken at multiple points along a rat's tail, for healthy mice (control group) and for healthy mice that had been dosed with a nitric oxide cream (GTNO) which is a known inhibitor of lymphatic performance. The graph in FIG. 4 shows that, while lymphatic pressure varies along the length of the tail, the slope of that line can be attributed to lymphatic health and performance, specifically, intrinsic contractility.

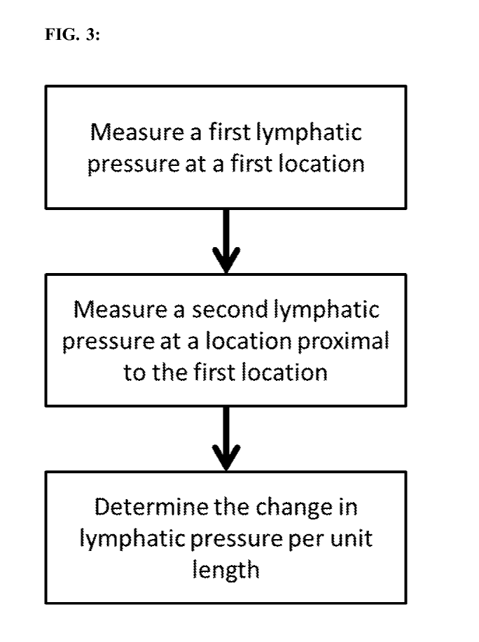

FIG. 4 graph showing PPL as an indicator of lymphatic performance. FIG. 4 shows the result of current research conducted by the inventors. Pressure measurements were taken at multiple points along a rat's tail, for healthy mice (control group) and for healthy mice that had been dosed with a nitric oxide cream (GTNO) which is a known inhibitor of lymphatic performance. The graph in FIG. 4 shows that, while lymphatic pressure varies along the length of the tail, the slope of that line can be attributed to lymphatic health and performance, specifically, intrinsic contractility.

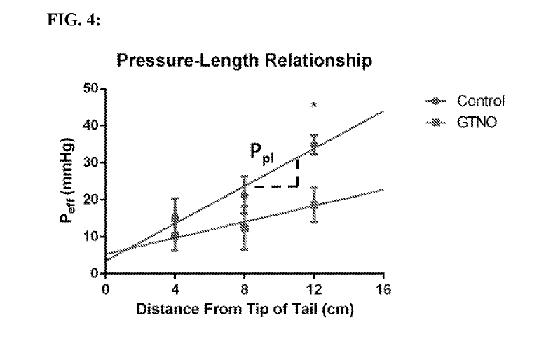

FIG. 5 illustrates the steps of the method for determining emptying rate.

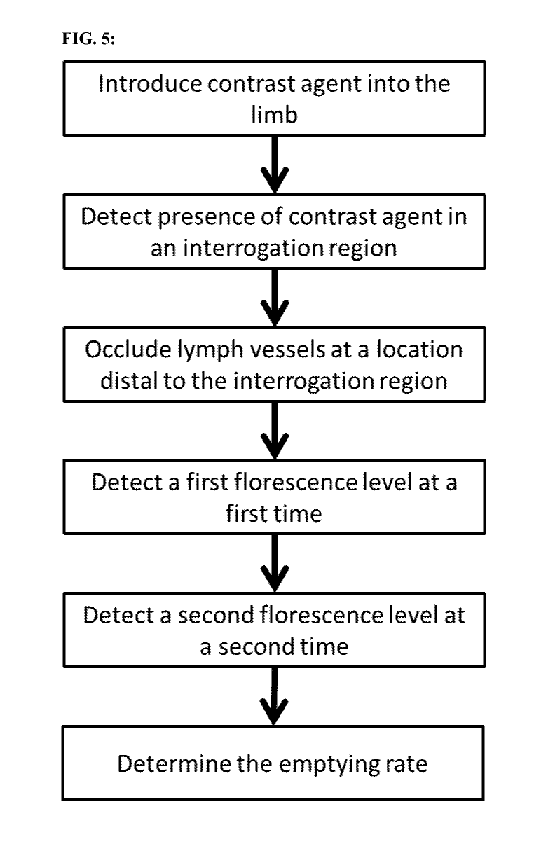

FIG. 6 that emptying rate provides a measure for determining reductions in lymphatic performance in mice. Healthy mice were used for the control, and healthy mice with a nitric oxide donor cream (GTNO) were used to illustrate mice with impaired lymphatics, as nitric oxide is a known inhibitor of lymphatic function.

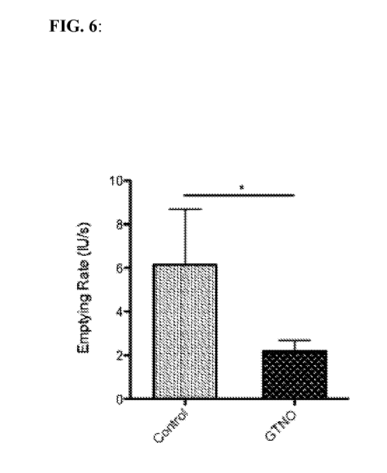

### DETAILED DESCRIPTION OF THE INVENTION

The present invention presents an apparatus capable of measuring lymphatic pressure, a system for performing the lymphatic pressure measurement, and methods for measuring lymphatic performance.

In exemplary embodiments, the present invention provides a method for measuring lymphatic performance, comprising the steps of (a) measuring a first lymphatic pressure at a first location on a limb of a mammal using a first measuring means, (b) measuring a second lymphatic pressure at a second location on the limb using a second measuring means, wherein the second location is disposed proximally from the first location and the distance between the first location and the second location is a separation distance, and (c) determining lymphatic performance by dividing the difference between the first lymphatic pressure and the second lymphatic pressure by the separation distance.

In exemplary embodiments, the method for measuring lymphatic performance comprises determining pressure change per unit length, emptying rate or a combination thereof.

I. Definitions The term “light source,” as used herein, refers to any device from which light primarily in the near infrared spectrum (750 nm-950 nm wavelength) is emitted. Lasers and light emitting diodes are typical examples. Other light sources that may also fall into this definition include edge-lit panels or optical fibers which are transmitting near infrared light from a remote source.

The term “light sensor,” as used herein, refers to any light sensing device which is sensitive to light in the near infrared spectrum, whether natively or with the addition of lenses and filters. CCD chips are a typical example, as well as photo diodes and avalanche photo diodes.

The term “contrast agent,” as used herein, refers to any flourophore which is excited by light in the near infrared spectrum and emits light within the near infrared spectrum. Examples of a contrast agent include indocyanine green, indocyanine green mixed with albumin, “800CW” produced by LICOR, and others.

The term “emptying rate,” as used herein, refers to the change in volume of lymph fluid within a given area over time. This may be approximated by occluding lymphatic flow in a region, determining the amount of lymph present in the region, and measuring the amount of time required for that amount of lymph to flow out of the region.

The term “lymphatic pressure measurement system,” as used herein, refers to any system that incorporates one or more light sources and light sensors, pressure cuffs, processors, and contrast agents to measure lymphatic pressure in a mammal. Such a system may include only one pressure cuff, or may have a plurality of cuffs that can be located along the same or different limbs to determine differential pressure measurements.

The term “integrated lymphatic pressure measurement system,” as used herein, refers to a lymphatic pressure measurement system wherein either the light sources, light sensors, or both, have been attached to or integrated with the pressure cuff such that they form a single unit.

The term “component lymphatic pressure measurement system,” as used herein, refers to a lymphatic pressure measurement system that is not an integrated lymphatic pressure measurement system.

The term “short time,” as used herein, refers to any time period between lymphatic pressure measurements such that a detectable amount of contrast agent remains in the region examined during the later occurring measurement. This time period depends on the volume of contrast agent introduced, the speed of lymphatic drainage, and the distance between measurement locations. As a result of taking measurements within a short time of each other, no additional contrast agent is required for the subsequent measurements. In exemplary embodiments, the time period is between 1 minutes and 10 minutes, 1 minute and 8 minutes, 1 minute and 6 minutes, 1 minute and 4 minutes, 1 minute and 3 minutes, 1 minute and 2 minutes, or less than one minute apart.

II. First Method for Measuring Lymphatic Performance

In exemplary embodiments, the present invention provides a method for measuring lymphatic performance by determining the pressure change per unit length (PPL) by measuring lymphatic pressure at two or more locations or points, for example, on the limb of a mammal. In exemplary embodiments, the method involves the various steps outlined below.

In exemplary embodiments, a first lymphatic pressure measurement is acquired by arranging an inflatable cuff around the circumference of a mammal's limb at a first location, introducing a contrast agent into the limb at location distal to the first location, detecting the presence of the contrast agent at an interrogation region proximal to or co-located with the first location by illuminating the interrogation region with one or more light sources and detecting the presence of the contrast agent with one or more light sensors, increasing the pressure in the cuff to occlude the flow of lymph, detecting an absence of contrast agent with the light sensors, incrementally decreasing the pressure in the cuff, detecting the return of contrast agent with the light sensors and identifying the pressure at that point to be the first lymphatic pressure. Optionally, while performing this first lymphatic pressure measurement, the emptying rate may also be calculated by acquiring a plurality of florescence measurements after the inflatable cuff has occluded the flow of lymph, and then calculating the change in florescence divided by the time between the measurements.

A second lymphatic pressure measurement is then acquired by arranging an inflatable cuff around the circumference of the limb at a second location which is proximal to the first location, detecting the presence of the contrast agent at an interrogation region proximal to or co-located with the second location by illuminating the interrogation region with one or more light sources and detecting the presence of the contrast agent with one or more light sensors, increasing the pressure in the cuff to occlude the flow of lymph, detecting an absence of contrast agent with the light sensors, incrementally decreasing the pressure in the cuff, detecting the return of contrast agent with the light sensors and identifying the pressure at that point to be the second lymphatic pressure. Lymphatic pressure change per unit length (PPL) is determined according to the following equation: (P2-P1)/|L2−L1|=PPL [pressure/length]; where P2 is the lymphatic pressure at the second location, P1 is the lymphatic pressure at the first location, L2-L1 is the distance between the first and second locations. By comparing lymphatic pressures acquired within a short period of time at two locations along the same limb, the extrinsic factors affecting lymphatic contractility are cancelled out. Thus, PPL is a metric that indicates intrinsic contractility of lymphatic vessels, which is thought to be the most important factor in the onset and progression of lymphedema.

The method may utilize a single inflatable cuff or two or more inflatable cuffs. When one inflatable cuff is utilized, the one or more additional pressure measurements are obtaining by moving or relocating the inflatable cuff to one or more additional cuff locations distinct from the cuff location of the first pressure measurement.

When two or more inflatable cuffs are utilized, they may be the same or different in terms of the configuration and/or components of the inflatable cuff.

Similarly, the one or more light sources and light sensors used for the first lymphatic pressure measurement may be different from the one or more light sources and light sensors used for the second lymphatic pressure measurement, or may be the same light sources and light sensors which are relocated after the first lymphatic pressure measurement is acquired.

The first emission wavelength and the second emission wavelength may be the same wavelength, in which case the first detection wavelength and the second detection wavelength will be the same wavelength, or the first emission wavelength and the second emission wavelength may be different wavelengths, in which case the first detection wavelength and the second detection wavelength will be different wavelengths. This is because the wavelength emitted by the contrast agent upon excitation is a function of the excitation wavelength, and since the emission wavelength may be the same or different, the wavelength emitted by the contrast agent may be the same or different, and thus the wavelength detected may be the same or different.

The distance between the first location and the second location may vary. In exemplary embodiments, the distance between the first location and the second location is between about 1 and about 18 inches, about 1 and about 16 inches, about 1 and about 14 inches, about 1 and about 12 inches, about 1 and about 10 inches, about 1 and about 8 inches, about 1 and about 6 inches, or about 1 and about 4 inches.

The present invention also encompasses an apparatus for measuring lymphatic pressure. In exemplary embodiments, the apparatus comprises one or more inflatable cuffs. In exemplary embodiments, the apparatus comprises two or more inflatable cuffs, which may be the same or different. Optionally, the apparatus comprises one or more inflatable cuffs integrated with one or more components selected from the group consisting of light source, light sensor, inflation means, deflation means, and pressure transducer.

The present invention also encompasses a system comprising an apparatus for measuring lymphatic pressure.

III. Second Method for Measuring Lymphatic Performance

The method for measuring lymphatic performance as indicated by the emptying rate of a lymphatic vessel. This method involves the various steps outlined below, as well as one or more optional or additional steps.

In exemplary embodiments, an emptying rate is acquired by arranging a lymphatic occlusion means around the circumference of a mammal's limb at a cuff location, introducing a contrast agent into the limb at location distal to the cuff location, detecting the presence of the contrast agent at an interrogation region proximal to or co-located with the cuff location by illuminating the interrogation region with one or more light sources and detecting the presence of the contrast agent with one or more light sensors, occluding the flow of lymph with the lymphatic occlusion means, acquiring a plurality of florescence measurements after the lymphatic occlusion means has occluded the flow of lymph, and then calculating the change in florescence divided by the time between the measurements. The emptying rate provides a measure of intrinsic contractility of lymphatic vessels that accounts for many but not all of the external factors that affect contractility. While PPL provides a better measure of intrinsic contractility, the emptying rate may be desirable for other reasons, such as its lower complexity or because it provides a spatially discrete measurement that can be compared over time.

The time between the two measurements may vary. In exemplary embodiments, the time is between about 5 and about 10 second, about 10 and about 20 seconds, about 20 and about 30 seconds, about 30 and about 40 seconds, about 40 and about 50 seconds, bout 50 and about 60 seconds, about 1 minute and about 90 seconds, about 90 seconds and about 2 minutes, about 2 minutes and about 180 seconds, about 180 seconds and about 3 minutes, or great than about 3 minutes.

The present invention also provides, in one embodiment, a lymphatic occlusion means, as well as a system comprising the lymphatic occlusion means and one or more additional components selected from a light source and a light sensor.

What is claimed is:

1. A method for measuring lymphatic performance, comprising the steps of
(a) measuring a first lymphatic pressure at a first location on a limb of a mammal using a first measuring means,
(b) measuring a second lymphatic pressure at a second location on the limb using a second measuring means, wherein the second location is disposed proximally from the first location and the distance between the first location and the second location is a separation distance, and
(c) determining lymphatic performance by dividing the difference between the first lymphatic pressure and the second lymphatic pressure by the separation distance.

2. The method of claim 1, wherein the first measuring means is a pressure cuff

3. The method of claim 1, wherein the first measuring means and the second measuring means are the same.

4. The method of claim 1, wherein the first measuring means and the second measuring means are different.

5. The method of claim 1, wherein step (b) is carried out within a short time of step (c).

6. The method of claim 1, wherein the first measuring means is an integrated lymphatic pressure measuring system.

7. The method of claim 1, wherein the first measuring means and the second measuring means are integrated lymphatic pressure measuring systems.

8. A method for measuring lymphatic performance by determining the change in pressure per unit length, comprising the steps of
(a) placing a first inflatable cuff around the circumference of a limb of a mammal at a first cuff location,
(b) illuminating said limb of a mammal with at least one first light source at a first interrogation region, wherein said first interrogation region is proximal to or co-located with said first cuff location, wherein said at least one first light source is operable to emit light at a first emission wavelength,
(c) imaging the first interrogation region with at least one first light sensor, wherein said at least one first light sensor is operable to detect light at a first detection wavelength,
(d) introducing a volume of a contrast agent into said limb of a mammal at an injection location, wherein said injection location is distal to said first cuff location, wherein said contrast agent is operable to emit light at said first detection wavelength when excited by light at said first emission wavelength,
(e) detecting the presence of florescence in the first interrogation region with the at least one first light sensor,
(f) inflating said first inflatable cuff to a pressure sufficient to block lymphatic flow,
(g) detecting the decrease in florescence in the first interrogation region with the at least one first light sensor,
(h) gradually deflating the first inflatable cuff,
(i) detecting the return of florescence in the first interrogation region with the at least one first light sensor,
(j) recording the pressure at which florescence returned as a first lymphatic pressure,
(k) placing a second inflatable cuff around the circumference of said limb of a mammal at a second cuff location, wherein the second cuff location is disposed at a separation distance proximal to the first cuff location,
(l) illuminating said limb of a mammal with at least one second light source at a second interrogation region, wherein said second interrogation region is proximal to or co-located with said second cuff location, wherein said at least one second light source is operable to emit light at a second emission wavelength,
(m) imaging the second interrogation region with at least one second light sensor, wherein said at least one second light sensor is operable to sense light at a second detection wavelength, such that the contrast agent emits light at the second detection wavelength when excited by light at the second emission wavelength,
(n) detecting the presence of florescence in the second interrogation region with the at least one second light sensor,
(o) inflating said second inflatable cuff to a pressure sufficient to block lymphatic flow,
(p) detecting the decrease in florescence in the second interrogation region with the at least one second light sensor,
(q) gradually deflating the second inflatable cuff,
(r) detecting the return of florescence in the second interrogation region with the at least one second light sensor,
(s) recording the pressure at which florescence returned as a second lymphatic pressure,
(t) calculating the change in pressure per unit length using the first lymphatic pressure, the second lymphatic pressure, and the separation distance.

9. The method of claim 8, wherein the second inflatable cuff and the first inflatable cuff are same.

10. The method of claim 8, wherein a single processor controls both the first inflatable cuff and the second inflatable cuff.

11. The method of claim 8, wherein the first emission wavelength is the same as the second emission wavelength.

12. The method of claim 8, wherein the at least one first light source and at least one first light sensor are attached to the first inflatable cuff

13. The method of claim 8, wherein the at least one second light source and at least one second light sensor are attached to the second inflatable cuff.

14. A method for measuring lymphatic performance by determining an emptying rate, comprising the steps of
(a) placing a lymphatic occlusion means around the circumference of a limb of a mammal at a cuff location,
(b) illuminating said limb of a mammal with at least one light source at an interrogation region, wherein said interrogation region is proximal to or co-located with said cuff location, wherein said at least one light source is operable to emit light within an emission range,
(c) imaging the interrogation region with at least one light sensor, wherein said at least one light sensor is operable to sense light within a detection range,
(d) introducing a volume of a contrast agent into said limb of a mammal at an injection location, wherein said injection location is distal to said cuff location, wherein said contrast agent is operable to emit light within said detection range when excited by light within said emission range,
(e) detecting the presence of florescence in the interrogation region with the at least one light sensor,
(f) occluding the lymphatics with the lymphatic occlusion means,
(g) determining a first level of florescence in the interrogation region with the at least one light sensor at a first time,
(h) determining a second level of florescence in the interrogation region with the at least one light sensor at a second time, and
(i) calculating an emptying rate by dividing the difference between the first level of florescence and the second level of florescence by the difference between the first time and the second time.
